(19) United States
(12) Patent Application Publication
(10) Pub. No.: US 2006/0241461 A1
(43) Pub. Date: Oct. 26, 2006

(54) System and method for 3-D visualization of vascular structures using ultrasound

(76) Inventors: Chris A. White, Toronto (CA); James I. Mehi, Thornhill (CA); Desmond Hirson, Thornhill (CA)

Correspondence Address: NEEDLE & ROSENBERG, P.C., SUITE 1000, 999 PEACHTREE STREET, ATLANTA, GA, 30309-3915, US

(21) Appl. No.: 11/395,534

(22) Filed: Mar. 31, 2006

Related U.S. Application Data
(60) Provisional application No. 60/667,376, filed on Apr. 1, 2005.

(51) Int. Cl. A61B 8/00

(52) U.S. Cl. 600/454

(57) ABSTRACT

A method for quantifying vascularity of a structure or a portion thereof comprises producing a plurality of two dimensional (2-D) high-frequency ultrasound image slices through at least a portion of the structure, wherein the structure or portion thereof is located within a subject, processing at least two of the plurality of 2-D ultrasound image slices to produce a three dimensional (3-D) volume image and quantifying the vascularity of the structure or portion thereof.

### CROSS-REFERENCE TO RELATED APPLICATIONS

This application claims the benefit of U.S. Provisional Application No. 60/667,376, filed on Apr. 1, 2005, which is herein incorporated by reference in its entirety.

### BACKGROUND

In many areas of biomedical research, accurately determining blood flow through a given organ or structure is critically important. For example, in the field of oncology, determination of blood flow within a tumor can enhance understanding of cancer biology and, since tumors need blood to grow and metastasize, determination of blood flow can help in the identification and the development of anti-cancer therapeutics. In practice, decreasing a tumor's vascular supply is often a primary goal of cancer treatment. To evaluate and develop therapeutics that affect the supply of blood to tumors, it is advantageous to quantify blood flow within tumors in small animal and in other subjects.

Typically, methods for determining the vascularity of structures within small animals have included histology based on sacrificed animal tissue. Also, Micro-CT of small animals allows imaging of organs to approximately 50 microns of resolution, but is lethal in most cases. While histology and Micro-CT provide accurate information regarding blood vessel structure, neither gives any indication as to in-vivo blood flow in the vessels. Therefore, histology and Micro-CT techniques are not ideal for the study of tumor growth and blood supply over time in the same small animal.

### SUMMARY

According to one embodiment of the invention, a method for quantifying vascularity of a structure or a portion thereof that is located within the a subject comprises producing a plurality of two dimensional (2-D) high-frequency “Power Doppler” or “Color Doppler” ultrasound image slices through at least a portion of the structure. In one aspect, at least two of the plurality of 2-D ultrasound image slices is processed to produce a three dimensional (3-D) volume image and the vascularity of the structure or portion thereof is quantified.

Other apparatus, methods, and aspects and advantages of the invention will be discussed with reference to the Figures and to the detailed description of the preferred embodiments.

### BRIEF DESCRIPTION OF THE FIGURES

The accompanying drawings, which are incorporated in and constitute a part of this specification, illustrate several aspects described below and together with the description, serve to explain the principles of the invention. Like numbers represent the same elements throughout the figures.

FIG. 1 is a block diagram illustrating an exemplary imaging system.

FIG. 2 shows an exemplary respiration waveform from an exemplary subject.

FIG. 3 shows an exemplary display of FIG. 1 with an exemplary color box of FIG. 1.

FIG. 4 is a block diagram illustrating an exemplary method of producing an ultrasound image using the exemplary system of FIG. 1.

FIG. 5 is a block diagram illustrating an exemplary method of producing an ultrasound image using the exemplary system of FIG. 1.

FIG. 6 is a block diagram illustrating an exemplary method of producing an ultrasound image using the exemplary system of FIG. 1.

FIGS. 7A and 7B are schematic diagrams illustrating exemplary methods of producing an ultrasound image slice using the exemplary system of FIG. 1.

FIG. 8 is a schematic diagram illustrating a plurality of two-dimensional (2-D) ultrasound image slices taken using the exemplary system of FIG. 1.

FIG. 9 is a schematic diagram of an ultrasound probe and 3-D motor of the exemplary system of FIG. 1, and a rail system that can be optionally used with the exemplary system of FIG. 1.

FIG. 10 is an exemplary 3-D volume reconstruction produced by the exemplary system of FIG. 1.

FIG. 11 is a block diagram illustrating an exemplary method of quantifying vascularity in a structure using the exemplary system of FIG. 1.

FIG. 12 is a flowchart illustrating the operation of the processing block of FIG.

FIG. 13 is a block diagram illustrating an exemplary array based ultrasound imaging system.

### DETAILED DESCRIPTION OF THE INVENTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a respiration waveform” can include two or more such waveforms unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The present invention may be understood more readily by reference to the following detailed description of preferred embodiments of the invention and the examples included therein and to the Figures and their previous and following description.

By a “subject” is meant an individual. The term subject includes small or laboratory animals as well as primates, including humans. A laboratory animal includes, but is not limited to, a rodent such as a mouse or a rat. The term laboratory animal is also used interchangeably with animal, small animal, small laboratory animal, or subject, which includes mice, rats, cats, dogs, fish, rabbits, guinea pigs, rodents, etc. The term laboratory animal does not denote a particular age or sex. Thus, adult and newborn animals, as well as fetuses (including embryos), whether male or female, are included.

According to one embodiment of the present invention, a method for quantifying vascularity of a structure or a portion thereof comprises producing a plurality of two dimensional (2-D) high-frequency Doppler ultrasound image slices through at least a portion of the structure. It is contemplated that the structure or portion thereof can be located within a subject. In operation, at least two of the plurality of 2-D ultrasound image slices is processed to produce a three dimensional (3-D) volume image and the vascularity of the structure or portion thereof is quantified.

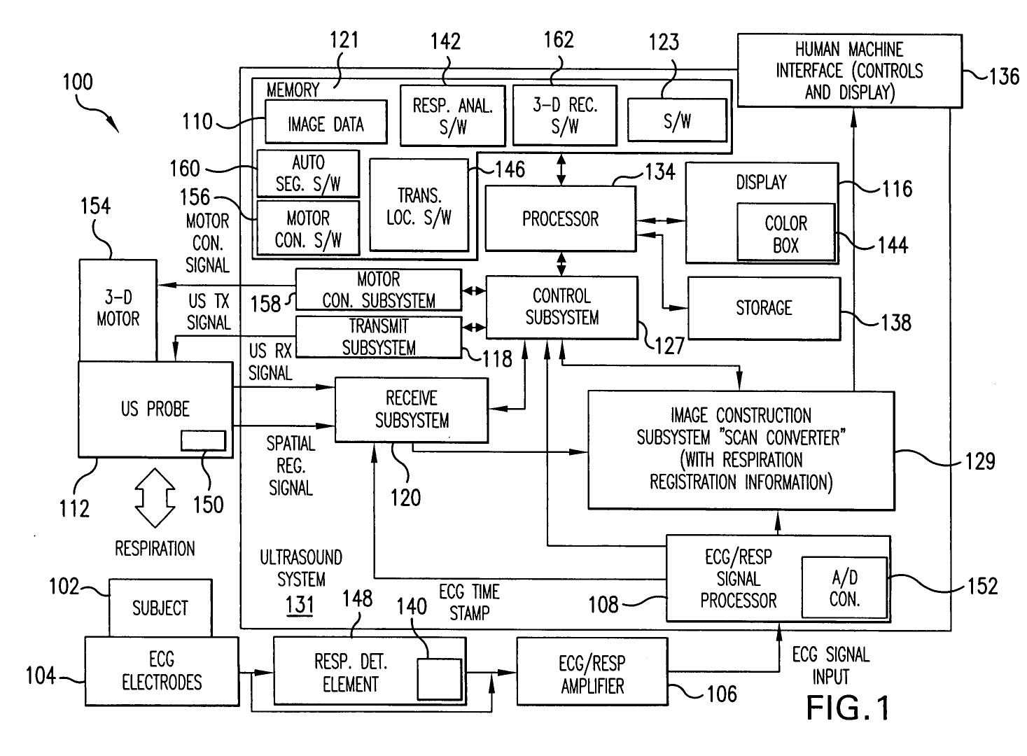

FIG. 1 is a block diagram illustrating an exemplary imaging system 100. The imaging system 100 operates on a subject 102. An ultrasound probe 112 is placed in proximity to the subject 102 to obtain ultrasound image information. The ultrasound probe can comprise a mechanically scanned transducer 150 that can be used for collection of ultrasound data 110, including ultrasound Doppler data. In the system and method described, a Doppler ultrasound technique exploiting the total power in the Doppler signal to produce color-coded real-time images of blood flow referred to as “Power Doppler,” can be used. The system and method can also be used to generate “Color Doppler” images to produce color-coded real-time images of estimates of blood velocity. The transducer can transmit ultrasound at a frequency of at least about 20 megahertz (MHz). For example, the transducer can transmit ultrasound at or above about 20 MHz, 30 MHz, 40 MHz, 50 MHz, or 60 MHz. Further, transducer operating frequencies significantly greater than those mentioned are also contemplated.

It is contemplated that any system capable of translating a beam of ultrasound across a subject or portion thereof could be used to practice the described methods. Thus, the methods can be practiced using a mechanically scanned system that can translate an ultrasound beam as it sweeps along a path. The methods can also be practiced using an array based system where the beam is translated by electrical steering of an ultrasound beam along the elements of the transducer. One skilled in the art will readily appreciate that beams translated from either type system can be used in the described methods, without any limitation to the type of system employed. Thus, one of skill in the art will appreciate that the methods described as being performed with a mechanically scanned system can also be performed with an array system. Similarly, methods described as being performed with an array system can also be performed with a mechanically scanned system. The type of system is therefore not intended to be a limitation to any described method because array and mechanically scanned systems can be used interchangeably to perform the described methods.

Moreover, for both a mechanically scanned system and an array type system, transducers having a center frequency in a clinical frequency range of less than 20 MHz, or in a high frequency range of equal to or greater than 20 MHz can be used.

In the systems and methods described, an ultrasound mode or technique, referred to as “Power Doppler” can be used. This Power Doppler mode exploits the total power in the Doppler signal to produce color-coded real-time images of blood flow. The system and method can also be used to generate “Color Doppler” images, which depict mean velocity information.

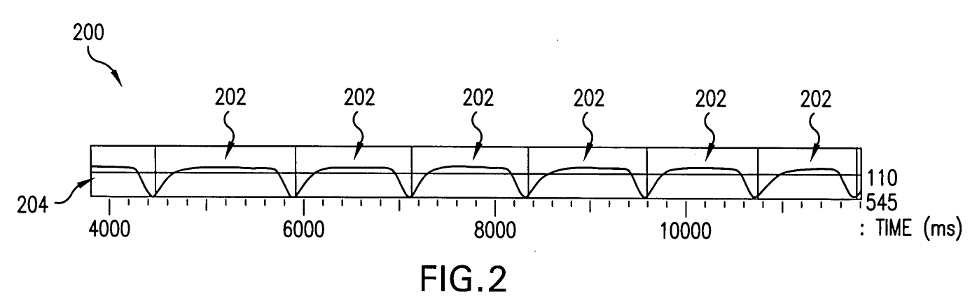

The subject 102 can be connected to electrocardiogram (ECG) electrodes 104 to obtain a cardiac rhythm and respiration waveform 200 (FIG. 2) from the subject 102. A respiration detection element 148, which comprises respiration detection software 140, can be used to produce a respiration waveform 200 for provision to an ultrasound system 131. Respiration detection software 140 can produce a respiration waveform 200 by monitoring muscular resistance when a subject breathes. The use of ECG electrodes 104 and respiration detection software 140 to produce a respiration waveform 200 can be performed using a respiration detection element 148 and software 140 known in the art and available from, for example, Indus Instruments, Houston, Tex. In an alternative aspect, a respiration waveform can be produced by a method that does not employ ECG electrodes, for example, with a strain gauge plethysmograph.

The respiration detection software 140 converts electrical information from the ECG electrodes 104 into an analog signal that can be transmitted to the ultrasound system 131. The analog signal is further converted into digital data by an analog-to-digital converter 152, which can be included in a signal processor 108 or can be located elsewhere, after being amplified by an ECG/respiration waveform amplifier 106. In one embodiment, the respiration detection element 148 comprises an amplifier for amplifying the analog signal for provision to the ultrasound system 131 and for conversion to digital data by the analog-to-digital converter 152. In this embodiment, use of the amplifier 106 can be avoided entirely. Using digitized data, respiration analysis software 142 located in memory 121 can determine characteristics of a subject's breathing including respiration rate and the time during which the subject's movement due to respiration has substantially stopped.

Cardiac signals from the electrodes 104 and the respiration waveform signals can be transmitted to an ECG/respiration waveform amplifier 106 to condition the signals for provision to an ultrasound system 131. It is recognized that a signal processor or other such device may be used instead of an ECG/respiration waveform amplifier 106 to condition the signals. If the cardiac signal or respiration waveform signal from the electrodes 104 is suitable, then use of the amplifier 106 can be avoided entirely.

In one aspect, the ultrasound system 131 comprises a control subsystem 127, an image construction subsystem 129, sometimes referred to as a scan converter, a transmit subsystem 118, a motor control subsystem 158, a receive subsystem 120, and a user input device in the form of a human machine interface 136. The processor 134 is coupled the control subsystem 127 and the display 116 is coupled to the processor 134.

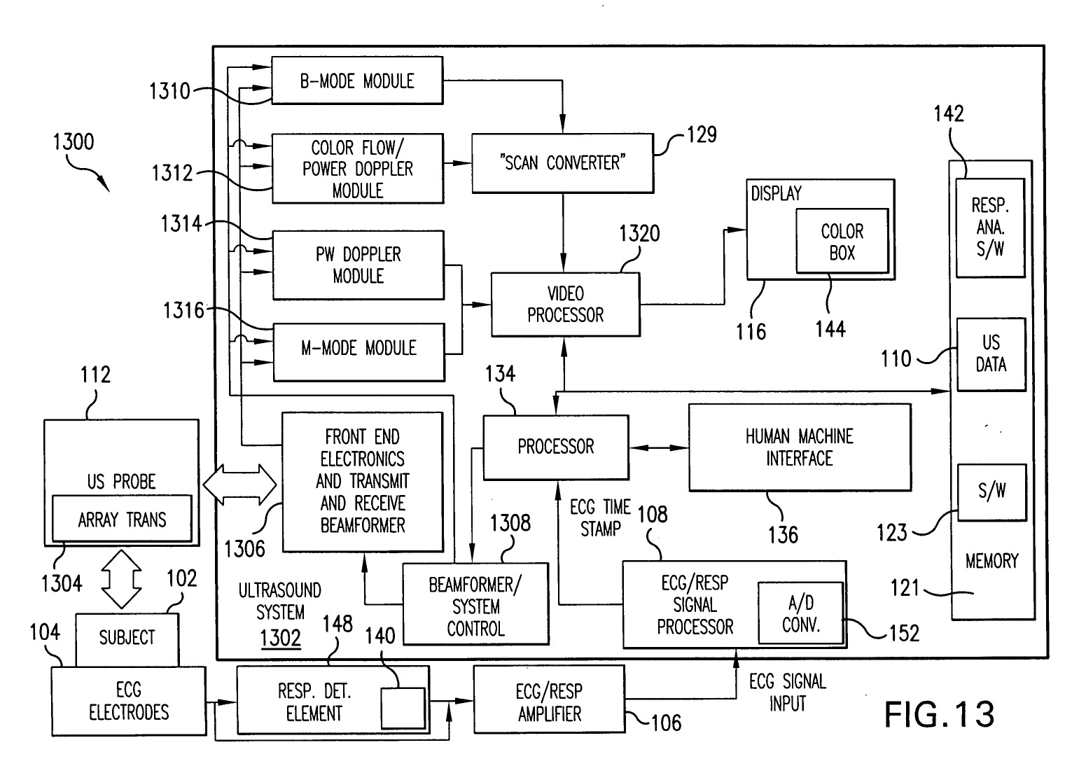

An exemplary ultrasound system 1302, as shown in FIG. 13, comprises an array transducer 1304, a processor 134, a front end electronics module 1306, a transmit beamformer 1306 and receive beamformer 1306, a beamformer control module 1308, processing modules Color flow 1312, and Power Doppler 1312, and other modes such as Tissue Doppler, M-Mode, B-Mode, PW Doppler and digital RF data, a scan converter 129, a video processing module 1320 a display 116 and a user interface module 136. One or more similar processing modules can also be found in the system 100 shown in FIG. 1.

A color box 144 can be projected to a user by the display 116. The color box 144 represents an area of the display 116 where Doppler data is acquired and displayed. The color box describes a region or predetermined area, within which Power Doppler or Color Doppler scanning is performed. The color box can also be generalized as a defining the start and stop points of scanning either with a mechanically moved transducer or electronically as for an array based probe.

The size or area of the color box 144 can be selected by an operator through use of the human machine interface 136, and can depend on the area in which the operator desires to obtain data. For example, if the operator desires to analyze blood flow within a given area of anatomy shown on the display 116, a color box 144 can be defined on the display corresponding to the anatomy area and representing the area in which the ultrasound transducer will transmit and receive ultrasound energy and data so that a user defined portion of anatomy can be imaged.

For a mechanically scanned transducer system, the transducer can be moved from the start position to the end position, such as, for example a first scan position through an nth scan position. As the transducer moves, ultrasound pulses are transmitted by the transducer and the return ultrasound echoes are received by the transducer. Each transmit/receive pulse cycle results in the acquisition of an ultrasound line. All of the ultrasound lines acquired as the transducer moves from the start to the end position constitute an image “frame.” For an ultrasound system which uses an array, the transmit beamformer, receive beamformer and front end electronics ultrasound pulses can be transmitted along multiple lines of sight within the color box. B-Mode data can be acquired for the entire field of view, whereas color flow data can acquired from the region defined by the color box.

In one exemplary aspect, the processor 134 is coupled to the control subsystem 127 and the display 116 is coupled to the processor 134. Memory 121 is coupled to the processor 134. The memory 121 can be any type of computer memory, and is typically referred to as random access memory “RAM,” in which the software 123 of the invention executes. Software 123 controls the acquisition, processing and display of the ultrasound data allowing the ultrasound system 131 to display an image.

The method and system for three-dimensional (3-D) visualization of vascular structures using high frequency ultrasound can be implemented using a combination of hardware and software. The hardware implementation of the system can include any or a combination of the following technologies, which are all well known in the art: discrete electronic components, discrete logic circuit(s) having logic gates for implementing logic functions upon data signals, an application specific integrated circuit having appropriate logic gates, a programmable gate array(s) (PGA), field programmable gate array (FPGA), and the like.

In one aspect, the software for the system comprises an ordered listing of executable instructions for implementing logical functions, and can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

In the context of this document, a “computer-readable medium” can be any means that can contain, store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM), a read-only memory (ROM), an erasable programmable read-only memory (EPROM or Flash memory) (magnetic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

The ultrasound system 131 software, comprising respiration analysis software 142, transducer localizing software 146, motor control software 156, and system software 123 determines the position of the transducer 150 and determines where to begin and end Power Doppler processing. For an exemplary array system, a beamformer control module controls the position of the scan lines used for Power Doppler, Color Flow, or for other scanning modalities.

The transducer localizing software 146 orients the position of the transducer 150 with respect to the color box 144. The respiration analysis software 142 allows capture of ultrasound data at the appropriate point during the respiration cycle of the subject 102. Thus, respiration analysis software 142 can control when ultrasound image data 110 is collected based on input from the subject 102 through the ECG electrodes 104 and the respiration detection software 140. The respiration analysis software 142 controls the collection of ultrasound data 110 at appropriate time points during the respiration waveform 200. In-phase (I) and quadrature-phase (Q) Doppler data can be captured during the appropriate time period when the respiration signal indicates a quiet period in the animal's breathing cycle. By “quiet period” is meant a period in the animal's respiratory or breathing cycle when the animal's motion due to breathing has substantially stopped.

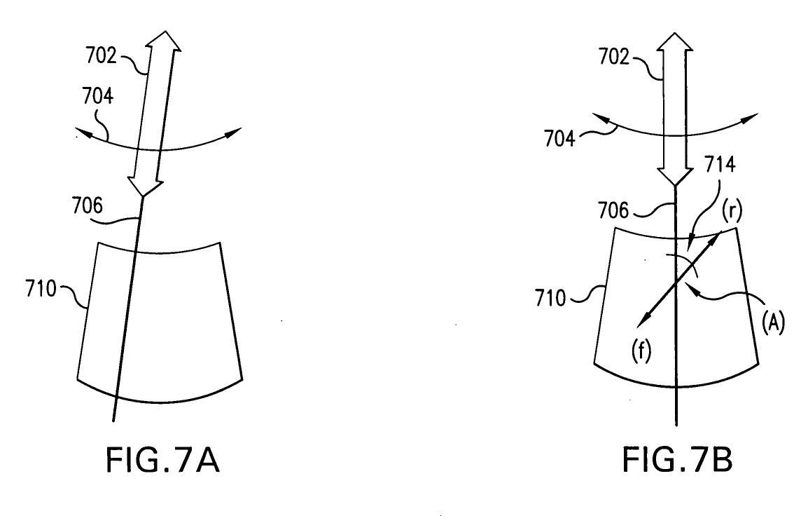

The motor control software 156 controls the movement of the ultrasound probe 112 along an axis (A) (FIG. 7B) so that the transducer 150 can transmit and receive ultrasound data at a plurality of locations of a subject's anatomy and so that multiple two-dimensional (2-D) slices along a desired image plane can be produced. Thus, in the exemplified system, the software 123, the respiration analysis software 142 and the transducer localizing software 146 can control the acquisition, processing and display of ultrasound data, and can allow the ultrasound system 131 to capture ultrasound images in the form of 2-D image slices (also referred to as frames) at appropriate times during the respiration waveform of the subject 200. Moreover, the motor control software 156, in conjunction with the 3-D motor 154 and the motor control subsystem 158, controls the movement of the ultrasound probe 112 along the axis (A) (FIG. 7B) so that a plurality of 2-D slices can be produced at a plurality of locations of a subject's anatomy.

Using a plurality of collected 2-D image slices the three dimensional (3-D) reconstruction software 162 can reconstruct a 3-D volume. The vascularity within the 3-D volume can be quantified using the 3-D reconstruction software 162 and auto-segmentation software 160 as described below.

Memory 121 also includes the ultrasound data 110 obtained by the ultrasound system 131. A computer readable storage medium 138 is coupled to the processor for providing instructions to the processor to instruct and/or configure the processor to perform algorithms related to the operation of ultrasound system 131, as further explained below. The computer readable medium can include hardware and/or software such as, by the way of example only, magnetic disk, magnetic tape, optically readable medium such as CD ROMs, and semiconductor memory such as PCMCIA cards. In each case, the medium may take the form of a portable item such as a small disk, floppy disk, cassette, or may take the form of a relatively large or immobile item such as a hard disk drive, solid state memory card, or RAM provided in the support system. It should be noted that the above listed example mediums can be used either alone or in combination.

The ultrasound system 131 comprises a control subsystem 127 to direct operation of various components of the ultrasound system 131. The control subsystem 127 and related components may be provided as software for instructing a general purpose processor or as specialized electronics in a hardware implementation. In another aspect, the ultrasound system 131 comprises an image construction subsystem 129 for converting the electrical signals generated by the received ultrasound echoes to data that can be manipulated by the processor 134 and that can be rendered into an image on the display 116. The control subsystem 127 is connected to a transmit subsystem 118 to provide ultrasound transmit signal to the ultrasound probe 112. The ultrasound probe 112 in turn provides an ultrasound receive signal to a receive subsystem 120. The receive subsystem 120 also provides signals representative of the received signals to the image construction subsystem 129. In a further aspect, the receive subsystem 120 is connected to the control subsystem 127. The scan converter 129 for the image construction subsystem and for the respiration registration information is directed by the control subsystem 127 to operate on the received data to render an image for display using the image data 110.

The ultrasound system 131 may comprise the ECG/respiration waveform signal processor 108. The ECG/respiration waveform signal processor 108 is configured to receive signals from the ECG/respiration waveform amplifier 106 if the amplifier is utilized. If the amplifier 106 is not used, the ECG/respiration waveform signal processor 108 can also be adapted to receive signals directly from the ECG electrodes 104 or from the respiration detection element 148. The signal processor 108 can convert the analog signal from the respiration detection element 148 and software 140 into digital data for use in the ultrasound system 131. Thus, the ECG/respiration waveform signal processor can process signals that represent the cardiac cycle as well as the respiration waveform 200. The ECG/respiration waveform signal processor 108 provides various signals to the control subsystem 127. The receive subsystem 120 also receives ECG time stamps or respiration waveform time stamps from the ECG/respiration waveform signal processor 108. For example, each data sample of the ECG or respiration data can be time registered with a time stamp derived from a clock.

In one aspect, the receive subsystem 120 is connected to the control subsystem 127 and an image construction subsystem 129. The image construction subsystem 129 is directed by the control subsystem 127. The ultrasound system 131 transmits and receives ultrasound data with the ultrasound probe 112, provides an interface to a user to control the operational parameters of the imaging system 100, and processes data appropriate to formulate still and moving images that represent anatomy and/or physiology of the subject 102. Images are presented to the user through the display 116.

The human machine interface 136 of the ultrasound system 131 takes input from the user and translates such input to control the operation of the ultrasound probe 112. The human machine interface 136 also presents processed images and data to the user through the display 116. Using the human machine interface 136 a user can define a color box 144. Thus, at the human machine interface 136, the user can define the color box 144 which represents the area in which image data 110 is collected from the subject 102. The color box 144 defines the area where the ultrasound transducer 150 transmits and receives ultrasound signals. Software 123 in cooperation with respiration analysis software 142 and transducer localizing software 146, and in cooperation with the image construction subsystem 129 operate on the electrical signals developed by the receive subsystem 120 to develop an ultrasound image which corresponds to the breathing or respiration waveform of the subject 102.

Using the human machine interface 136, a user can also define a structure or anatomic portion of the subject for the 3-D visualization of vascular structures within that structure or anatomic portion of the subject. For example, the user can define the overall size, shape, depth and other characteristics of a region in which the structure to be imaged is located. These parameters can be input into the ultrasound system 131 at the human machine interface 136. The user can also select or define other imaging parameters such as the number of 2-D ultrasound slices that are produced and the spacing between each 2-D slice. Using these input parameters, the motor control software 156 controls the movement of the 3-D motor 154 and the ultrasound probe 112 along the defined structure or portion of the subject's anatomy. Moreover, based on the separation between and absolute number of 2-D slices produced, the auto-segmentation software 160 and the 3-D reconstruction software 162 can reconstruct a 3-D volume of the structure or portion of anatomy. The structure's or anatomic portion's vascularity percentage can be determined by the 3-D reconstruction software 162 or by the system software 123 as described below.

FIG. 2 shows an exemplary respiration waveform 200 from a subject 102 where the x-axis represents time in milliseconds (ms) and the y-axis represents voltage in millivolts (mV). A typical respiration waveform 200 includes multiple peaks or plateaus 202, one for each respiration cycle of the subject. As shown in FIG. 2, a reference line 204 can be inserted on the waveform 202. The portions of the respiration waveform 200 above the reference line 204, are peaks or plateaus 202, and generally represent the period when the subject's movement due to breathing has substantially stopped, i.e., a “motionless” or “non-motion” period. One skilled in the art will appreciate that what is meant by “substantially stopped” is that a subject's movement due to breathing has stopped to the point at which the collection of Doppler ultrasound data is desirable because of a reduction in artifacts and inaccuracies that would otherwise result in the acquired image due to the breathing motion of the subject.

It is to be understood that depending on the recording equipment used to acquire respiration data and the algorithmic method used to analyze the digitized signal, the motionless period may not align perfectly with the detected signal position. Thus, time offsets can be used that are typically dependent on the equipment and detection method used and animal anatomy. For example, in one exemplary recording technique that uses the muscular resistance of the foot pads, the motionless period starts shortly after the detected peak in resistance. It is contemplated that the determination of the actual points in the respiration signal, regardless of how it is acquired, can be determined by empirical comparison of the signal to the actual animal's motion and choosing suitable corrections such that the signal analysis performed can produce an event describing the respective start and stop points of the respiration motion.

A subject's motion due to breathing substantially stops for a period of approximately 100 to 2000 milliseconds during a respiration cycle. The period during a subject's respiration cycle during which that subject's motion due to breathing has substantially stopped may vary depending on several factors including, animal species, body temperature, body mass or anesthesia level. The respiration waveform 200 including the peaks 202 can be determined by the respiration detection software 140 from electrical signals delivered by ECG electrodes 104 which can detect muscular resistance when breathing. For example, muscular resistance can be detected by applying electrodes to a subject's foot pads.

By detecting changes in muscular resistance in the foot pads, the respiration detection software 140 can generate the respiration waveform 200. Thus, variations during a subject's respiration cycle can be detected and ultrasound data can be acquired during the appropriate time of the respiration cycle when the subject's motion due to breathing has substantially stopped. For example, Doppler samples can be captured during the approximately 100 milliseconds to 600 millisecond period when movement has substantially ceased. A respiration waveform 200 can also be determined by the respiration detection software 140 from signals delivered by a pneumatic cushion (not shown) positioned underneath the subject. Use of a pneumatic cushion to produce signals from a subject's breathing is known in the art.

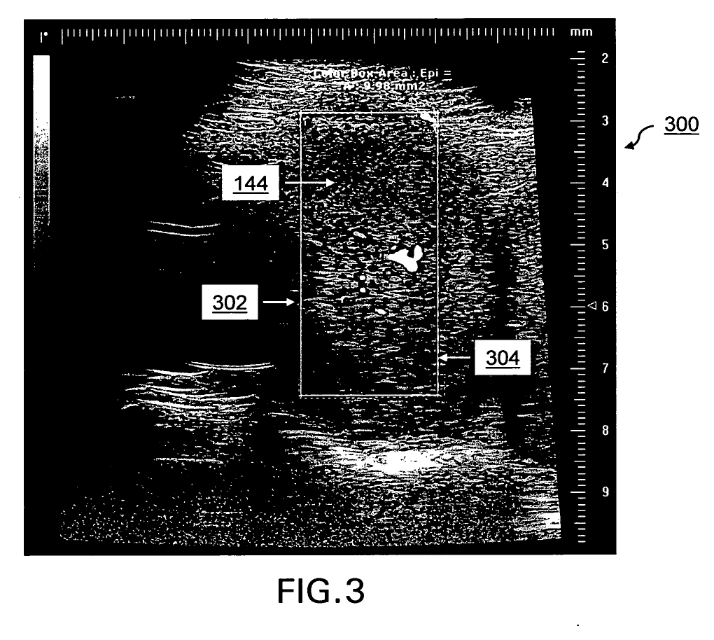

FIG. 3 shows an exemplary display 116 of the ultrasound imaging system 131 with an exemplary color box 144. The image 300 represents an image displayed on the display 116. The color box 144 is defined within the image 300. The color box 144 represents an area of the ultrasound image 300 on the display 116 that corresponds to a portion of the subject's anatomy where ultrasound data is collected by the ultrasound probe 112. As will be understood to one skilled in the art, multiple color boxes 144 can also be defined simultaneously on the display or at different times and such multiple color boxes 144 can be used in the methods described.

The area encompassed by the color box 144 can be defined by a user via the human machine interface 136 or configured automatically or semi-automatically based on a desired predefined image size such as field of view (FOV). Thus, the color box 144 represents an area where data is captured and depicted on the display 116. The image data 110 is collected within the color box 144 by registering the transducer 150 of the ultrasound probe 112 within the color box 144. The ultrasound transducer 150 can be a single element sweeping transducer. The ultrasound transducer 150 can be located anywhere on the anatomy that corresponds to a defined color box 144. The transducer localizing software 146 can be used to localize the transducer 150 at any defined location within the color box 144.

The initial position of the transducer 150 can define a starting point for transmitting and receiving ultrasound energy and data. Thus, in one example, the transducer 150 can be located at the left side 302 of the color box 144 and ultrasound energy and data can be transmitted and received starting at the left side of the color box. Similarly, any portion of the color box 144 can be defined as an end point for transmitting and receiving ultrasound energy and data. For example, the right side 304 of the color box 144 can be defied as an end point for transmitting and receiving ultrasound energy and data. Ultrasound energy and data can be transmitted and received at any point and time between the starting and end point of the color box. Therefore, in one aspect of the invention, a user can define the left side 302 of a color box 144 as the starting point and the right side 304 of the same color box 144 as an end point. In this example, ultrasound energy and data can be transmitted and received at any point and time between the left side 302 of the color box 144 and moving towards the right side 304 of the color box 144. Moreover, it would be clear to one skilled in the art that any side or region of a color box 144 could be defined as the starting point and any side or region of a color box 144 could be defined as an end point.

It is to be understood by one skilled in the art that all references to motion using a mechanically positioned transducer are equally applicable to suitable configuration of the beamformer in an array based system and that these methods described herein are applicable to both systems. For example, stating that the transducer should be positioned at its starting point is analogous to stating that the array beamformer is configured to receive ultrasound echoes at a start position.

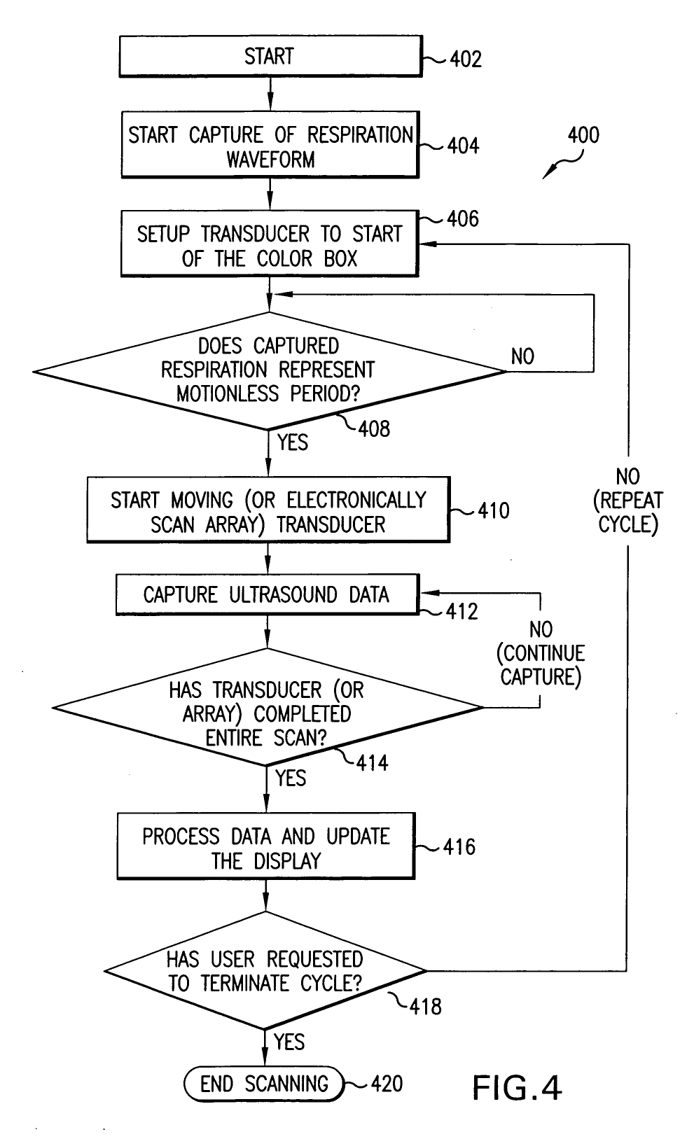

FIG. 4 is a flowchart illustrating an exemplary method of producing one or more 2-D ultrasound image slice (FIG. 7A, B) using the exemplary imaging system 100 or exemplary array system 1300. As would be clear to one skilled in the art, and based on the teachings above, the method described could be performed using an alternative exemplary imaging system.

At a start position 402, a single element transducer 150 or an array transducer 1304 is placed in proximity to a subject 102. In block 404, a respiration waveform 200 from the subject 102 is captured by respiration detection software 140. In one aspect, the respiration waveform 200 is captured continuously at an operator selected frequency. For example, the respiration waveform can be digitized continuously at 8000 Hz. In block 406, once the transducer 150 is placed in proximity to the subject 102, the transducer is positioned at a starting position in the color box 144. In one embodiment, the transducer is positioned at the left side 302 of the color box 144 when the color box is viewed on the display 116. However, any side or region of a color box could be defined as the starting point and any side or region of a color box could be defined as an end point.

In step 408, the respiration analysis software 142 determines if a captured sample represents the start of the motionless period 202 of the respiration waveform 200. One skilled in the art will appreciate that the point at which the motionless or non-motion period begins is not necessarily the “peak” of the respiratory waveform; also, the point in the waveform which corresponds to the motionless period can be dependent on the type of method used to acquire the respiratory waveform. A captured sample of the continuously captured respiration waveform 200 represents the value of the captured respiration waveform 200 at a point in time defined by the selected sampling frequency. At a particular point 202 of the subject's respiration waveform 100, the subject's movement due to breathing has substantially stopped. This is a desired time for image data to be captured. As noted above, a mechanically moved transducer or an array transducer can be used for collection of ultrasound data.

Prior to the initialization of Color Flow, or Power Doppler scanning, the transducer can be positioned at the start point defined by the color box. In block 410, if respiration analysis software 142 determines that the subject 102 is at a point which represents the beginning of the motionless period 202 of its respiration cycle, the transmit subsystem 118 under the control of the software 123 causes the transducer 150 to start moving. If the captured sample at block 406 does not represent a “peak” 202 of the subject's respiration cycle, the respiration detection software 142 continues to monitor for a respiration peak 202.

In block 412, the transducer begins scanning and ultrasound data is acquired. For a mechanically scanned transducer system, the speed of motion can be set such that it completes the entire scan from start to stop within the motionless period of the respiration cycle. In block 414, the completion of the frame is checked. If frame completion has not occurred, the process loops back to block 412, and scanning continues. If the completion of frame has occurred, then scanning stops, the data is processed and the display is updated in block 416. After the display has been updated, in block 418 the system software checks for a user-request to terminate imaging. In block 420, if the image termination request has occurred, imaging stops. If, in block 418, no termination request has been made, the process loops back to block 406.

The period of time during which ultrasound samples are captured can vary depending on the subject's respiration cycle. For example, ultrasound samples can be collected for a duration of between about 200 to about 2000 milliseconds. Ultrasound I and Q data can be captured during the quiet period in the subject's respiration cycle for Doppler acquisition. Envelope data can be acquired for B-Mode. For example, 200 milliseconds is an estimate of the period of time which a subject 102 may be substantially motionless in its respiration cycle 200. This substantially motionless period is the period when the ultrasound samples are collected.

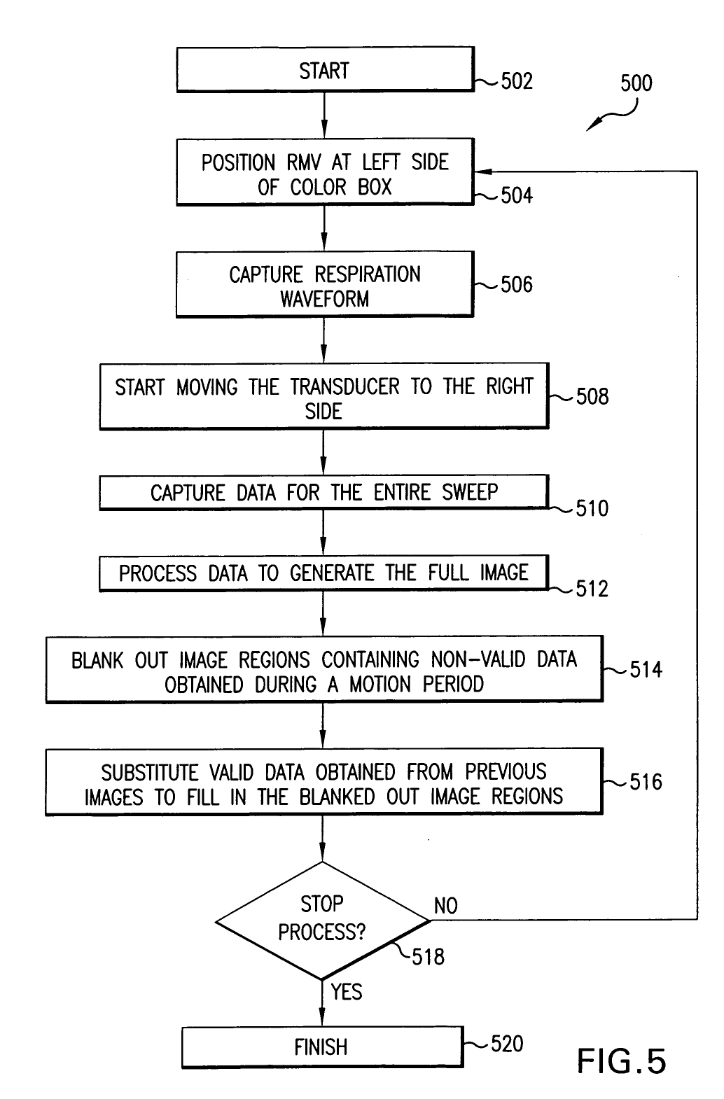

FIG. 5 is a flowchart 500 illustrating an alternative method of producing an image using the exemplary imaging system 100 or array system 1300. As will be clear to one skilled in the art, and based on the teachings above, the method described could be performed using an alternative exemplary imaging system. The method 500 uses the same hardware as the method 400 and can use respiration analysis software 142 and transducer localizing software 146 programmed according to the noted modes and methodologies described herein. As with method outlined in flowchart 400, the transducer can be positioned at the left side 302 of the color box 144. Or, in the case of an array based system, the beamformer can be configured to begin scanning at the left side of the color box. It will be clear to one skilled in the art that any side or region of a color box could be defined as the starting point and any side or region of a color box could be defined as an end point.

In block 504, the transducer is placed at the left side 302 of the color box. In block 506, a respiration waveform is captured. The respiratory waveform can be time stamped, such that there is known temporal registration between the acquired ultrasound lines and the respiratory waveform. This form of scanning involves time registration of the respiratory waveform. A new frame can be initiated as soon as the previous one ends. Therefore, the respiratory waveform and the start of frame may not be synchronous. The time period during which maximum level of respiratory motion occurs, the motion period, is determined from the respiratory waveform using the respiratory analysis software. Data which is acquired during this time period is assumed to be distorted by respiratory motion and is termed “non-valid” data. Data acquired during the motionless phase of the respiratory cycle is termed “valid” data. In various exemplary aspects, the non-valid data can be replaced with valid data from the same region acquired during a previous frame, or with data obtained by processing valid data acquired during previous frames using an averaging or persistence method.

In block 508, software 123 causes the transducer to start moving to the right side 304 of the color box and performs a complete sweep of the color box.

It is contemplated that a mechanically moved transducer 150 or an array transducer 1304 can be used for collection of ultrasound data. In block 510, ultrasound data is captured for the entire sweep or translation across the color box 508. In block 512, the data is processed to generate an initial data frame comprising B-mode data and Doppler data. In block 514, the respiratory waveform is processed to determine the “blanked period,” which corresponds to the period during which there is high respiratory motion in the subject and the regions of the image lines within the frame, which occurred during the “blanked period” are determined from the time stamp information. These lines which were acquired during the “blanked period” are not displayed. Instead the lines in the blanked region are filled in. There are various methods which can be used to fill in the blanked regions. For example, previously acquired frames can be stored in a buffer in memory, and the video processing software can display lines from previously acquired frames which correspond to the blanked out lines. Thus, in block 516, data from a previous data frame can be used to fill in areas blanked out in block 514.

In one exemplary aspect, the process for producing an ultrasound image outlined in FIG. 5 comprises monitoring a respiration waveform of a subject and detecting at least one peak period and at least one non-peak period of the respiration waveform. In this aspect, each peak period corresponds to a time when the subject's bodily motion caused by its respiration has substantially stopped and each non-peak period corresponds to a time when the subject's body is in motion due to its respiration. The process further comprises generating ultrasound at a frequency of at least 20 megahertz (MHz), transmitting ultrasound at a frequency of at least 20 MHz into a subject, and acquiring ultrasound data during the least one peak period of the subject's respiration waveform and during the at least one non-peak period of the subject's respiration waveform. In exemplary aspects, the steps of generating, transmitting and acquiring are incrementally repeated from a first scan line position through an nth scan line position.

In this example, the received ultrasound data are complied to form an initial data frame comprising B-mode and Doppler data. At least one portion of the initial data frame comprising data received during a non-peak period of the subjects respiration waveform is identified and processed to produce a final data frame. In this aspect, the final data frame is compiled from data received during the incremental peak periods of the subject's respiration waveform.

In aspects of this example, the processing step comprises removing data, i.e., “non-valid” data, from an initial data frame that was received during non-peak periods of the subject's respiration waveform to produce a partially blanked out data frame having at least one blanked out section and substituting data, i.e., “valid” data, received during the peak of the subject's respiration waveform from another initial data frame into the at least one blanked out region of the partially blanked out data frame to produce an ultrasound image. The substituted data received during the peak of the subject's respiration waveform can be from a region of its data frame that spatially corresponds to the blanked out region of the partially blanked out region of the partially blanked out image. For example, a line take at a specific location along the transducer arc spatially corresponds to a second line taken at that same location along the transducer arc. Such corresponding lines, groups of lines or regions can be taken while motion due to breathing has substantially stopped or while motion due to breathing is present. Regions taken during periods where the animal's movement due to breathing has substantially stopped can be used to substitute for corresponding regions taken during times when the animal's movement due to breathing is not substantially stopped.

In one aspect, persistence can be applied to color flow image data. As one skilled in the art will appreciate, persistence is a process in which information from each spatial location in the most recently acquired frame is combined according to an algorithm with information from the corresponding spatial locations from previous frames. In one aspect, persistence processing may occur in the scan converter software unit. An exemplary persistence algorithm that can be processed is as follows:
Y(n)=αY(n−1)+(1−a)X(n),
where Y(n) is the output value which is displayed, X(n) is the most recently acquired Power Doppler sample, Y(n−1) is the output value derived for the previous frame, and a is a coefficient which determines the amount of persistence. When there are non-valid or blanked regions in the most recently acquired image frame, persistence can be applied to the entire frame, with the non-valid lines being given a value of zero. Provided that the start of frame of each Power Doppler frame is not synchronous with the respiratory waveform, the non-valid time periods occurs at different times within each frame.

Another exemplary method of handling the non-valid or blanked regions is to implement persistence on a line to line basis. For lines which have a valid value, persistence is implemented as above. For lines which are determined to be within the non-valid region, the persistence operation is suspended. Thus, in the above equation, instead of setting X(n) to zero and calculating Y(n), Y(n) is set equal to Y(n−1).

In block 518, it is determined whether to stop the process. In one aspect, the condition to stop the process is met when the position of the transducer meets or exceeds the stop position of the color box 144. In an alternative aspect, the process can continue until an operator issues a stop command. If, in block 518, it is determined that the process is not complete, the transducer is repositioned at the left side 302 of the color box. If in block 518, it is determined that the process is finished, the process is complete at block 520. The blanking process described in block 514 and 516 is optional. In some cases, if for example the rate at which the transducer moves across the anatomy is high, the entire data set may be acquired without a respiration event occurring. In these cases, image or frame blanking is not performed.

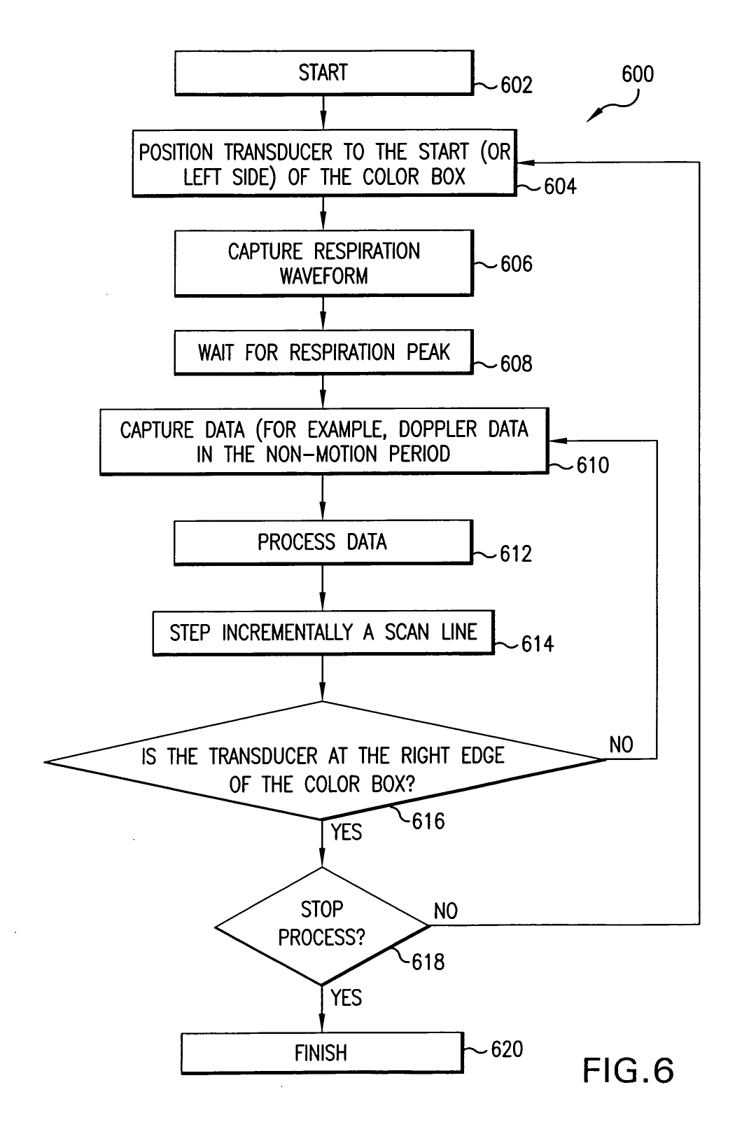

FIG. 6 is a flow chart illustrating a third exemplary embodiment 600 for producing one or more 2-D image slice (FIG. 7A, B) using the imaging system 100. As will be clear to one skilled in the art, and based on the teachings above, the method described could be performed using an alternative exemplary imaging system. In this method, the transducer 150 is moved once per respiration cycle. A mechanically scanned transducer can be used for collection of ultrasound data. Thus, in this method, one line of data is captured when the subject's movement due to respiration has substantially stopped. Once this substantially motionless period ends, the transducer recaptures image data the next time in the subject's respiration cycle when the subject is substantially motionless again. Thus, one line of data is captured per respiration cycle when the subject is substantially still.

The method 600 begins at block 602. In block 604, a transducer is positioned at the start of the color box 144. In one example, the left side 302 of the color box 144 can be defined as start point for the transducer and the right side 304 can be defined as the end point. In block 606, a respiration waveform is captured from the subject 102 using ECG electrodes 104 and respiration detection software 140. In block 608, respiration analysis software 142 analyzes the respiration waveform and instructs the ultrasound system 131 to wait for a respiration peak 202.

In block 610, Doppler samples are captured in the quiet time of the respiration wave approximately 100 to 2000 milliseconds after the respiration peak detected in block 608. The quiet period depends on the period of the subject's respiration. For example, in a mouse, the quiet period can be approximately 100 to 2000 milliseconds. Doppler I and Q data can be captured during the quiet period in the animal's respiration cycle. In block 612, captured ultrasound Doppler data is processed by the ultrasound system 131, and in block 614 a step motor moves the transducer 150 a small distance through the color box 144. In block 616, it is determined whether the transducer is at the end 304 of the color box 144. If it is determined that the transducer is not at the end 304 of the color box 144, a line of Doppler data is captured during a peak 202 of the respiration waveform. If it is determined that the transducer is at the right edge 304 of the color box, it is further determined at block 618 whether to stop the process. If the transducer is at the right edge 304 of the color box the process is stopped. If it is determined that the process is to be stopped, the process is finished. If it is determined that the process is not finished because the transducer is not at the right edge 304 of the color box, the transducer is repositioned to the start or left side 302 of the color box.

FIGS. 7A and 7B are schematic representations depicting methods of ultrasound imaging using a plurality of 2-D images slices produced using the methods described above. As shown in FIG. 7A, the ultrasound probe 112 transmits an ultrasound signal in a direction 702 projecting a “line” 706 of ultrasound energy. The ultrasound probe 112 pivots and/or a mechanically scanned transducer within the probe sweeps along an arc 704 and propagates lines of ultrasound energy 706 originating from points along the arc. The ultrasound transducer thus images a two dimensional (2-D) plane or “slice” 710 as it moves along the arc 704. Alternatively, if an array is used, the ultrasound beam is swept across a 2-D plane by steering or translation by electronic means, thus imaging a 2-D “slice”.

A 2-D slice is considered to be the set of data acquired from a single 2-D plane through which the ultrasound beam is swept or translated one or more times. It may consist of one or more frames of B-Mode data, plus one or more frames of color flow Doppler data, where a frame is considered to be the data acquired during a single sweep or translation of the ultrasound beam.

FIG. 7B illustrates an axis (A) that is substantially perpendicular to a line of energy 706 projected at the midpoint of the arc 704. The ultrasound probe can be moved along the axis (A). To move the ultrasound probe 112 along the axis (A), the imaging system 100 uses a “3-D Motor” 154, which receives input from the motor control subsystem 158. The motor 154 can be attached to the ultrasound probe 112 and is capable of moving the ultrasound probe 112 along the axis (A) in a forward (f) or reverse (r) direction. The ultrasound probe 112 is typically moved along the axis (A) after a first 2-D slice 710 is produced. To move the ultrasound probe along the axis (A) so that a plurality of image slices can be produced, the imaging system 100 or an array system 1300 can further comprise an integrated multi-rail imaging system as described in U.S. patent application Ser. No. 11/053,748 titled “Integrated Multi-Rail Imaging System” filed on Feb. 7, 2005, which is incorporated herein in its entirety.

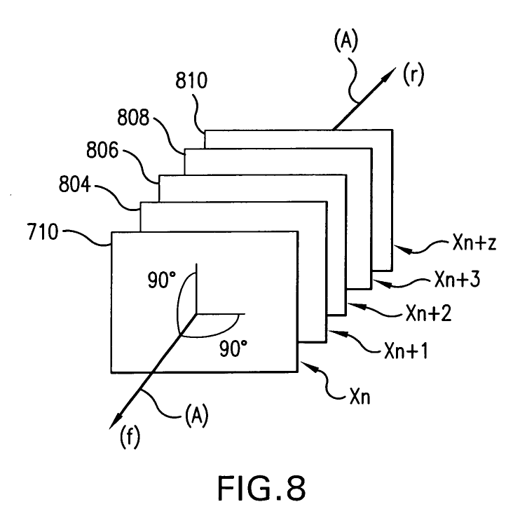

FIG. 8 is a schematic representation illustrating that a first 2-D slice 710 can be produced at a position Xn. Moreover, at least one subsequent slice 804 can be produced at a position Xn+1. Additional slices can be produced at positions Xn+2 (806), Xn+3 (808) and at Xn+z (810). Any of the 2-D slices can be produced using the methods described above while the subject's movement due to breathing has substantially stopped.

To move the ultrasound probe 112 along the axis (A) at the appropriate time, the motor control subsystem 158 receives signals from the control subsystem 127, which, through the processor 134, controls movement of the 3-D motor 154. The motor control system 158 can receive direction from motor control software 156, which allows the ultrasound system 131, to determine when a sweep of the probe 112 has been competed and a slice has been produced, and when to move the ultrasound probe 112 along the axis (A) to a subsequent point for acquisition of a subsequent slice at a subsequent position. An exemplary system, such as system 1300, can also be used. A motor can be used to move an array transducer or a probe comprising an array transducer along the axis (A). Similarly to that for the single element transducer system, the system can determine when a slice has been taken with the array and when to move the transducer or a probe comprising the transducer along the axis (A) to a next location.

The motor control software 156 can also cause the motor to move the ultrasound probe 112 a given distance along the axis (A) between each location Xn where ultrasound is transmitted and received to produce a 2-D slice. For example, the motor control software 156 can cause the 3-D motor 154 to move the ultrasound probe 112 about 50 microns (em) along the axis (A) between each 2-D slice produced. The distance between each 2-D slice can be varied, however, and is not limited to 50 μm. For example, the distance between each slice can be about 1.0 μm, 5 μm, 10 μm, 50 μm, 100 μm, 500 μm, 1000 μm, 10,000 μm, or more.

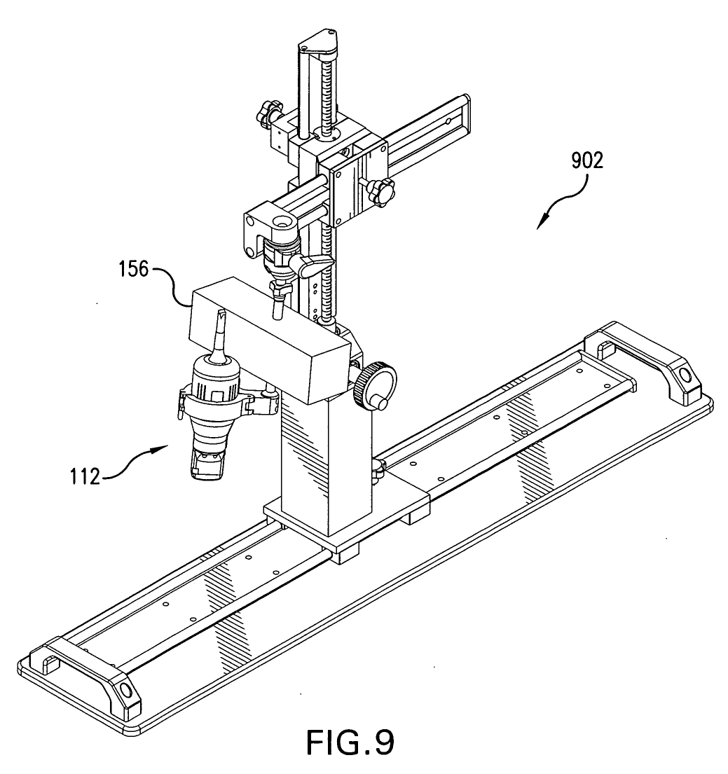

As described above, the number of slices produced and the distance between each slice can be defined by a user and can be input at the human machine interface 136. Typically, the 3-D motor 156 is attached to a rail system 902 (FIG. 9) that allows the motor 154 and ultrasound probe 112 to move along the axis (A). In one aspect, the 3-D motor 154 is attached to both the ultrasound probe 112 and the rail system 902.

Once the ultrasound probe 112 has been moved to a next position on the axis (A), a subsequent 2-D slice 804 at position Xn+1 can be produced by projecting a line of ultrasound energy from the transducer 150 along an arc similar to arc 704, but in a new location along the axis (A). Once the 2-D slice 804 has been produced, the ultrasound probe 112 can be moved again along the axis (A), and a subsequent slice 806 at position Xn+2 can be produced. Each 2-D slice can be produced using the methods described above while the subject's movement due to breathing has substantially stopped. Each slice produced can be followed by movement of the probe in a forward (f) or reverse (r) direction along the axis (A).

The sequence of producing a 2-D ultrasound image slice and moving the probe 112 can be repeated as many times as desired. For example, the ultrasound probe 112 can be moved a third time, and a fourth ultrasound image slice 808 at a position Xn+3 can be produced, or the probe can be moved for a z number time and a slice 810 at a position Xn+z, can be produced. The number of times the sequence is repeated depends on characteristics of the structure being imaged, including its size, tissue type, and vascularity. Such factors can be evaluated by one skilled in the art to determine the number of 2-D slices obtained.

Each two dimensional slice through a structure or anatomic portion that is being imaged generally comprises two primary regions. The first region is the area of the structure where blood is flowing. The second region is the area of the structure where blood is not flowing. If the imaged structure is a tumor, this second region generally comprises the parenchyma and supportive stoma of the tumor and the first region comprises the blood flowing through the vascular structures of the tumor. The vascularity of a structure (i.e. a tumor) can be determined by quantifying blood flow.

At least two 2-D slices can be combined to form an image of a three dimensional (3-D) volume. Because the 2-D slices are separated by a known distance, for example 50 μm, the 3-D reconstruction software 162 can build a known 3-D volume by reconstructing at least 2 two-dimensional slices.

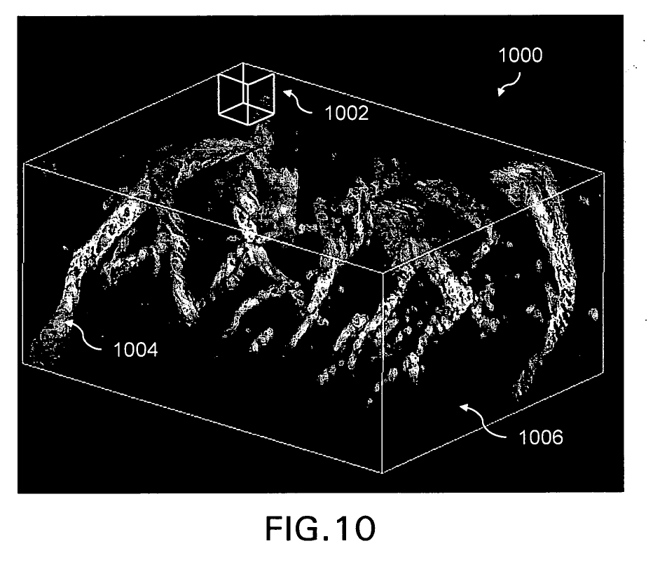

FIG. 10 is a schematic view showing an exemplary 3-D volume 1000 produced by combining at least two 2-D image slices. The 3-D volume 1000 comprises a volume of a vascular structure or a portion thereof. The boundary of the volume of the structure can be defined to reconstruct the three dimensional volume of the structure or portion thereof. The boundary can be defined by an autosegmentation process using autosegmentation software 160. Autosegmentation software 160 (Robarts Research Institute, London, Ontario, Canada) and methods of using autosegmentation software 150 to determine the structure boundary are known in the art. Generally, autosegmentation software 160 follows the grey scale contour and produces the surface area and volume of a structure such as a tumor. It is contemplated that this autoselected region can be alternatively manually selected and/or refined by the operator. The same or alternative software know in the art can be used to reconstruct the three dimensional volume of the structure or portion thereof after the boundary is defined. Subsequent determination and analysis of voxels as described below, can be performed on voxels within the defined or reconstructed structure volume.

Because a plurality of 2-D slices is combined to produce the 3-D volume 1000, the 3-D volume comprises the same two primary regions as the 2-D slices. The first region 1004 is the region where blood is flowing within the imaged structure or portion thereof, which can be displayed as a color flow Doppler image. The second region 1006, is where blood is not flowing within the imaged structure or portion thereof.

Once the 3-D volume 1000 is produced, a voxel 1002 can be superimposed within the 3-D volume using the 3-D reconstruction software 162 and using methods known in the art. Voxels 1002 are the smallest distinguishable cubic representations of a 3-D image. The full volume of the 3-D volume 1000 can be divided into a number of voxels 1002, each voxel having a known volume. The total number of voxels can be determined by the 3-D reconstruction software 162.

When the 3-D volume 1000 is divided into voxels 1002, each voxel is analyzed by the 3-D reconstruction software 162 for color data, which represents blood flow. In one exemplary aspect, Power Doppler can represent blood flow power as color versus a grey scale B-mode image. For example, if the ultrasound system displays fluid or blood flow as the color red, then each red voxel represents a portion of the 3-D volume where blood is flowing.

Each colored voxel within the structure is counted and a total number of colored voxels (Nᵥ) is determined by the 3-D reconstruction software 162. A threshold discriminator can be used to determine whether a colored voxel qualifies as having valid flow. The threshold can be determined automatically, or can be calculated automatically based on analysis of the noise floor of the Doppler signal. The threshold can also be a user adjustable parameter. The 3-D reconstruction software 162 multiplies Nᵥ by the known volume of a voxel (Vᵥ) to provide an estimate of the total volume of vascularity (TVᵥₐₛ) within the entire 3-D volume. Thus, TVᵥₐₛ=Nᵥ*Vᵥ. The total volume of vascularity can be interpreted as an estimate of the spatial volume occupied by blood vessels in which there is flow detectable by Power Doppler processing. The 3-D reconstruction software 162 can then calculate the percentage vascularity of a structure, including a tumor, by dividing TVᵥₐₛ by the total volume of the structure (TVₛ). The total volume of the structure can be calculated by multiplying the total number of voxels within the structure (Nₛ) by the volume of each voxel (V_{V}). Thus, TV_{S}=Nᵥ*Vᵥ, and percentage vascularity =(Nᵥ*Vᵥ)/(Nₛ*Vᵥ). It can be seen that the term Vᵥ cancels, therefore percentage vascularity =Nᵥ/Nₛ.

Thus, provided herein is a method for determining the percentage vascularity of a vascular structure or portion thereof. The method comprises determining the total volume (TVₛ) and the total volume of vascularity (TVᵥₐₛ) of the structure or portion thereof using ultrasound imaging. The method further comprises determining the ratio of TVᵥₐₛ to TVₛ, wherein the ratio of TVᵥₐₛ to TVₛ provides the percentage vascularity of the structure or portion thereof.

In one aspect, the TVₛ of the structure or portion thereof is determined by producing a plurality of two dimensional ultrasound slices taken through the structure or portion thereof. Each slice can be taken at location along an axis substantially perpendicular to the plane of the slice and each slice being separated by a known distance along the axis. B-mode data is captured at each slice location, a three dimensional volume of the structure or portion thereof is reconstructed from the B-mode data captured at two or more slice locations, and the TVₛ is determined from the reconstructed three dimensional volume. The determination of the three dimensional volume of the structure can comprise first determining the surface contour or boundary using automated or semi-automated procedures as described herein.

The TVᵥₐₛ of the structure or portion thereof can be determined by capturing Doppler data at each slice location. The Doppler data represents blood flow within the structure or portion thereof. The number of voxels within the reconstructed three dimensional volume that comprise captured Doppler data are quantified and the number of voxels comprising Doppler data are multiplied by the volume of a voxel to determine the TVᵥₐₛ. Since a slice may contain one or more frames of Doppler data, averaging of frames within a slice or the application of persistence to the frames within a slice may be used to improve the signal to noise ratio of the Doppler data.

In an alternate implementation the magnitude of the Power Doppler signal of the voxels can be used to calculate a value which is proportional to the total blood flow within the 3-D volume. In this implementation the 3-D reconstruction software 162 sums the magnitude of the Power Doppler signal of each voxel in the image (Pᵥ). The parameter Pᵥ may be multiplied by a parameter Kᵥ prior to summation. Thus TP=ΣPᵥ*Kᵥ, where the summation is carried out over the number of voxels containing flow. A threshold discriminator may be used to qualify valid flow. Since the magnitude of the Power Doppler signal is proportional to the number of red blood cells in the sample volume, TP becomes a relative measure of the volume of vasculature. The parameter Kᵥ may be proportional to the volume of each voxel. Compensation for variations in signal strength may also be incorporated into Kᵥ. For example, variations in signal strength with depth may arise from tissue attenuation, or from the axial variation of the intensity of the ultrasound beam. Kᵥ can provide a correction factor for a particular voxel. The correction factor can provide compensation for effects such as depth dependent variations in signal strength due to tissue attenuation, and variations in the axial intensity of the ultrasound beam.

TVₛ can be determined by an autosegmentation process using autosegmentation software 160. Autosegmentation software 160 (Robarts Research Institute, London, Ontario, Canada) and methods of using autosegmentation software 150 to determine the total volume of a structure (TVₛ) are known in the art. Generally, autosegmentation software 160 follows the grey scale contour and produces the surface area and volume of a structure such as a tumor. It is contemplated that this autoselected region can be alternatively manually selected and/or refined by the operator.

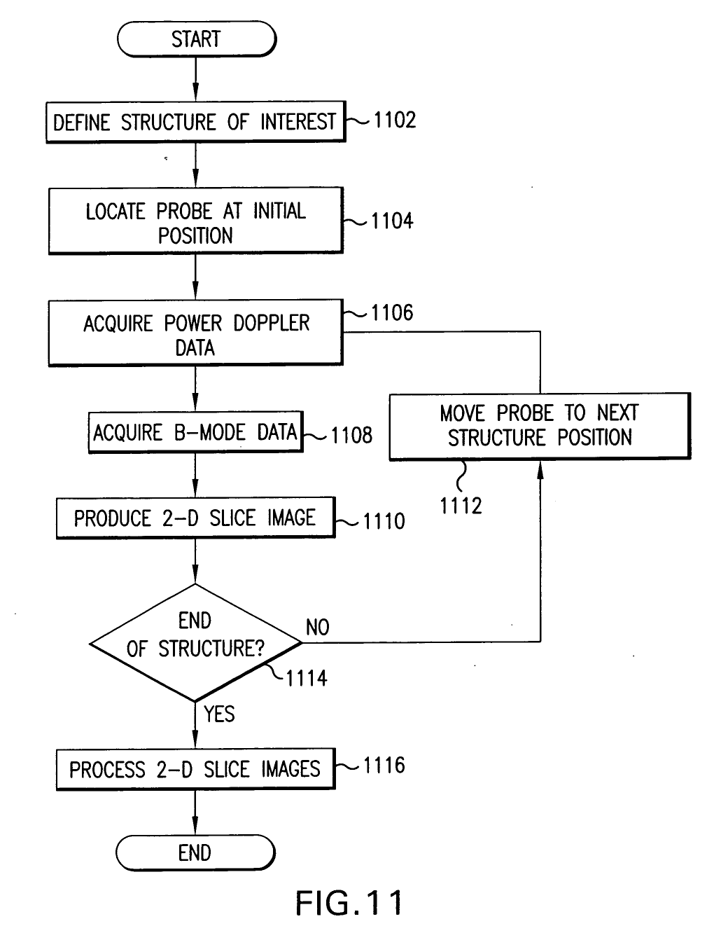

FIG. 11 is a block diagram illustrating an exemplary method 1100 of producing an ultrasound image using the exemplary imaging system 100. In block 1102, a structure of interest is defined. The structure can be defined by a user at the human machine interface 136. In one embodiment, the defined structure is a tumor, or a portion thereof, which can be located within a small animal subject. As used throughout, a structure means any structure within a subject, or portion thereof that has blood flowing through it. A structure can be an entire tumor in a subject, or a portion of that tumor. The structure can also be an organ or tissue, or any portion of that organ or tissue with blood flowing through it. The structure is typically located in a subject. Software can be used to define the structure of interest. For example, the autosegmentation software 160 can be used to define a structure of interest. Moreover, imaging modalities including but not limited to ultrasound, radiography, CT scanning, OCT scanning, MRI scanning, as well as, physical exam can also be used to define a desired structure for imaging using the described methods.

In block 1104, a single element transducer 150 is placed in proximity to a subject 102 and the ultrasound probe 112 is located at an initial position. This position corresponds to a portion of the structure of interest at which ultrasound imaging begins. It can also correspond to a position in proximity to the structure of interest at which ultrasound imaging begins.

In block 1106, the transducer 150 transmits ultrasound and receives Power Doppler ultrasound data. Using the methods described above, ultrasound energy can be transmitted and received when the subject's movement due to breathing has substantially stopped. A mechanically scanned ultrasound transducer 150 can be used for collection of ultrasound data. Doppler samples are captured and collected as the transducer 150 sweeps, or the probe 112 pivots, across an arc. More than one Power Doppler frame may be acquired in order to allow blanked out regions to be filled in.

In block 1108, the transducer 150 transmits ultrasound and receives B-mode ultrasound data. Using the methods described above, ultrasound energy can be transmitted and received when the subject's movement due to breathing has substantially stopped. This additional B-Mode frame is spatially aligned with the Power Doppler overlay, and therefore can act as a reference frame for the Power Doppler data acquired previously. The additional B-Mode frame provides anatomical and reference information.

In block 1110, the data collected in block 1106 and 1108 is used to produce a composite 2-D slice image consisting of a Doppler image overlayed onto the acquired B-Mode frame. If in block 1114 it is determined that the previously acquired slice was not the final slice in the structure, in block 1112 the probe is moved to the next structure position along axis (A). If, in block 1114, it is determined that this slice was the last slice in the defined structure then the structure has been fully imaged. Whether a structure is “fully imaged” can be determined by the user or can be based on user input parameters, or characteristics of the imaged structure. For example, the structure may be fully imaged when a certain number of slices have been produced through the full extent of a defined structure or portion thereof or when the end of a color box 144 is reached.

If, in block 1114, it is determined that the defined structure has been fully imaged, the 2-D slices produced are processed in block 1116. If, in block 1114, it is determined that the defined structure has not been fully imaged, then the probe is moved to a next position in block 1112, data is acquired again in block 1106 and a subsequent slice is produced in block 1110.

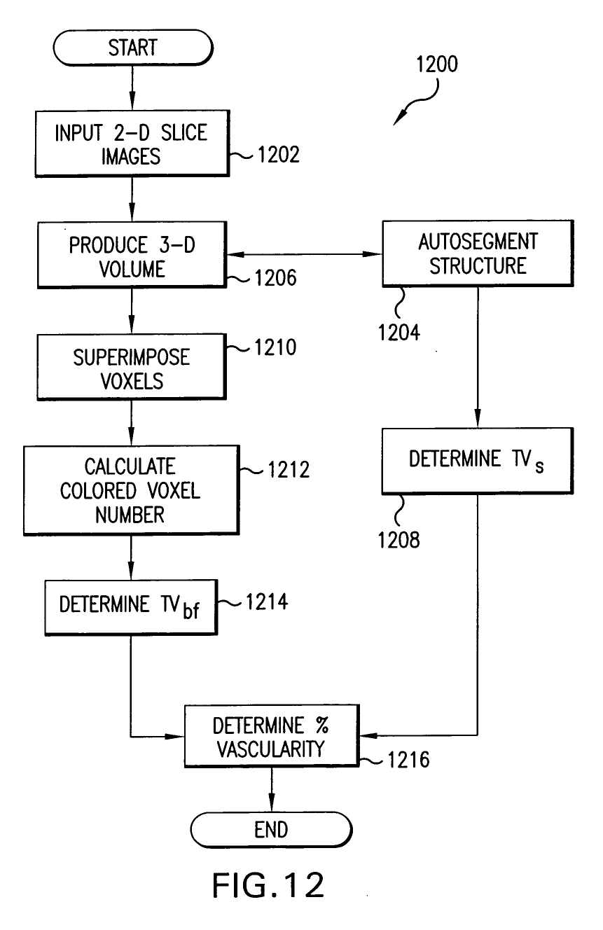

FIG. 12 is a flow chart illustrating the “PROCESS 2-D SLICE IMAGES” block 1116 of FIG. 11. In block 1202, the 2-D slice images produced in block 1108 of FIG. 11 are input into the 3-D reconstruction software 162. In block 1206, a 3-D volume is produced from the 2-D image slices using the 3-D reconstruction software 162. In block 1210, voxels are superimposed throughout the 3-D volume using the 3-D reconstruction software 162. In block 1212, the 3-D reconstruction software 162 calculates the total number of colored voxels within the 3-D volume. In block 1214, the total volume of voxels with color (representing blood flow) TVᵥₐₛ is determined by multiplying the total number of colored voxels by the known volume of a voxel.

In block 1204, the autosegmentation software 160 determines the surface area of the structure of interest within the 3-D volume. In block 1208, the total volume of the structure of interest TVₛ is determined.

In block 1216, the vascularity percentage of the structure of interest is determnined. The vascularity percentage can be determined by dividing the total volume of voxels having blood flow TVᵥₐₛ determined in block 1208 by the total volume of the structure of interest TVₛ determined in block 1214.

The preceding description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. The corresponding structures, materials, acts, and equivalents of all means or step plus function elements in the claims below are intended to include any structure, material, or acts for performing the functions in combination with other claimed elements as specifically claimed.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification. The blocks in the flow charts described above can be executed in the order shown, out of the order shown, or substantially in parallel.

Accordingly, those who work in the art will recognize that many modifications and adaptations to the present invention are possible and can even be desirable in certain circumstances and are a part of the present invention. Other embodiments of the invention will be apparent to those skilled in the art from consideration of the specification and practice of the invention disclosed herein. Thus, the preceding description is provided as illustrative of the principles of the present invention and not in limitation thereof. It is intended that the specification and examples be considered as exemplary only, with a true scope and spirit of the invention being indicated by the following claims.

What is claimed is:

1. A method for determining the percentage vascularity of a vascular structure or portion thereof, comprising:
determining the total volume (TVₛ) and the total volume of vascularity (TVᵥₐₛ) of the structure or portion thereof using ultrasound imaging; and
determining the ratio of TVᵥₐₛ to TVₛ, wherein the ratio of TVᵥₐₛ to TVₛ provides the percentage vascularity of the structure or portion thereof.

2. The method of claim 1, wherein the TVₛ of the structure or portion thereof is determined by:
producing a plurality of two dimensional ultrasound slices taken through the structure or portion thereof, each slice being taken at location along an axis substantially perpendicular to the plane of the slice and each slice being separated by a known distance along the axis;
capturing B-mode data at each slice location;
reconstructing a three dimensional volume of the structure or portion thereof from the B-mode data captured at two or more slice locations; and
determining the TV, from the reconstructed three dimensional volume.

3. The method of claim 2, wherein the TVᵥₐₛ of the structure or portion thereof is determined by:
capturing Doppler data at each slice location, the Doppler data representing blood flow within the structure or portion thereof,
quantifying the number of voxels within the reconstructed three dimensional volume that comprise captured Doppler data and multiplying the number of voxels comprising Doppler data by the volume of a voxel to determine the TVᵥₐₛ.

4. The method of claim 2, wherein the TVᵥₐₛ of the structure or portion thereof is determined by:
capturing Doppler data at each slice location, the Doppler data representing blood flow within the structure or portion thereof,
quantifying the number of voxels within the reconstructed three dimensional volume that do not comprise captured Doppler data;
multiplying the number of voxels not comprising Doppler data by the volume of a voxel; and
subtracting the determined multiple from the determined TVₛ to determine the TVᵥₐₛ.

5. The method of claim 3, wherein each voxel that has a measured power that is less than a predetermined threshold value is disregarded in the calculation of TVᵥₐₛ.

6. The method of claims 3 or 4, further comprising determining the total power of the blood flow within the structure or portion thereof.

7. The method of claim 6, wherein the total power of the blood flow within the structure or portion thereof is determined by the summation of the product of the Power Doppler value of each voxel with a parameter Kᵥ, wherein Kᵥ provides a correction factor for depth dependent signal variation.

8. The method of claim 7, wherein each voxel that has a measured power that is less than a predetermined threshold value is disregarded.

9. The method of claim 3, wherein the captured Doppler data is Power Doppler data.

10. The method of claim 3, wherein the captured Doppler data is Color flow Doppler data.

11. The method of claim 3, wherein the structure is located within a subject.

12. The method of claim 11, wherein the captured Doppler data and the B-mode data are produced using ultrasound transmitted into the subject or portion thereof at a frequency of 20 MHz or higher.

13. The method of claim 11, wherein the subject is a small animal.

14. The method of claim 13, wherein the small animal is selected from the group consisting of a mouse, rat, and rabbit.

15. The method of claim 11, wherein the structure is a tumor.

16. The method of claim 3, wherein each location along the axis corresponds to a predefined area of a portion of the subject's anatomy where the B-mode data and Doppler data is captured from the subject.

17. The method of claim 3, wherein the structure is located within a subject and wherein the B-mode data and the Doppler data are captured when the subject's movement due to breathing has substantially stopped.

18. The method of claim 17, further comprising:
monitoring a respiration waveform of a subject and detecting a peak period in the waveform, wherein the peak corresponds to a time when the subject's bodily motion caused by its respiration has substantially stopped;
capturing the B-mode data and Doppler data from the subject, wherein the capturing is performed during the waveform peak period corresponding to the time when the subject's bodily motion caused by its respiration has substantially stopped.

19. The method of claim 18, further comprising, prior to the step of capturing the B-mode data and Doppler data from the subject,
generating ultrasound at a frequency of at least 20 megahertz (MHz); and
transmitting ultrasound at a frequency of at least 20 MHz into the subject, wherein the steps of generating, transmitting and capturing are performed during the waveform peak period corresponding to the time when the subject's bodily motion caused by its respiration has substantially stopped.

20. The method of claim 19, wherein the steps of generating, transmitting and capturing are incrementally repeated at each location along the axis to capture the B-mode data and the Doppler data.

21. The method of claim 17, further comprising:
monitoring a respiration waveform of a subject and detecting at least one peak period in the respiration waveform, each peak period corresponding to a time when the subject's bodily motion caused by its respiration has substantially stopped, and at least one non-peak period of the respiration waveform, each non-peak period corresponding to a time when the subject's body is in motion due to its respiration;
generating ultrasound at a frequency of at least 20 megahertz (MHz);
transmitting ultrasound at a frequency of at least 20 MHz into a subject;
capturing the B-mode data and Doppler data from the subject during the least one peak period of the subject's respiration waveform and during the at least one non-peak period of the subject's respiration waveform, wherein the steps of generating, transmitting and capturing are incrementally repeated at each location along the axis;
compiling the captured ultrasound data at each slice location to form an initial data frame comprising a B-mode data and Doppler data;
identifying at least one portion of the initial data frame comprising data received during a non-peak period of the subject's respiration waveform;
processing the initial data frame to produce a final data frame for each slice location, wherein the final data frame is compiled from B-mode and Doppler data received during the incremental peak periods of the subject's respiration waveform; and
reconstructing the three dimensional volume from a plurality of final data frames.

22. The method of claim 21, wherein the processing step comprises:
removing data from the initial data frame that was received during non-peak periods of the subject's respiration waveform at location along the axis to produce a partially blanked out data frame having at least one blanked out region; and
substituting data received during the peak of the subject's respiration waveform from at least one other initial data frame taken at the same location along the axis into the at least one blanked out region of the partially blanked out image to produce the final date frame.

23. The method of claim 22, wherein the substituted data received during the peak of the subject's respiration waveform is from a region of its data frame that spatially corresponds to the blanked out region of the partially blanked out region of the partially blanked out image.

24. A system for determining the percentage vascularity of a vascular structure or portion thereof, comprising:
a transducer for generating ultrasound at a frequency of at least 20 MHz, for transmitting at least a portion of the generated ultrasound into the vascular structure or portion thereof, and for capturing ultrasound energy; and
a processor for determining the total volume (TVₛ) and the total volume of vascularity (TVᵥₐₛ) of the structure or portion thereof from the captured ultrasound energy and for determining the ratio of TVᵥₐₛ to TVₛ, wherein the ratio of TVᵥₐₛ to TVₛ provides the percentage vascularity of the structure or portion thereof.

25. The system of claim 24, further comprising means for monitoring a respiration waveform of a subject and for detecting a peak period in the waveform, wherein the peak corresponds to a time when the subject's bodily motion caused by its respiration has substantially stopped.

26. The system of claim 24, wherein the processor is configured for determining the total power of the blood flow within the vascular structure or portion thereof.
